(19) United States
(12) Patent Application Publication
(10) Pub. No.: US 2003/0114798 A1
(43) Pub. Date: Jun. 19, 2003

(54) Medicament delivery device

(75) Inventors: Christopher Nigel Langley, Warwickshire (GB); Robert Frederick Veasey, Warwickshire (GB)

Correspondence Address: OLIFF & BERRIDGE, PLC, P.O. BOX 19928, ALEXANDRIA, VA, 22320, US

(73) Assignee: DCA DESIGN INTERNATIONAL LIMITED, Warwick (GB)

(21) Appl. No.: 10/318,085

(22) Filed: Dec. 13, 2002

(30) Foreign Application Priority Data
Dec. 18, 2001 (GB) 0130140.7

(51) Int. Cl. A61M 5/24

(52) U.S. Cl. 604/184; 604/200

(57) ABSTRACT

The present invention relates to a medicament delivery device used for self-administration of a medicament regimen by a patient. In such medicament delivery devices the administration of the medicament is from a replaceable medicament cartridge located within a housing forming a part of the medicament delivery device. Removal of the cartridge for those with infirmities who are not able to grip the cadge sufficiently firmly to place or remove the cartridge is improved by the present invention. A medicament delivery device is disclosed comprising a main housing and a secondary housing, the secondary housing being pivotally connected at a first end to the main housing, the secondary housing being movable between an operative position in which the secondary housing is adjacent along its length to the main housing and a loading position in which the secondary housing is pivoted away from the main body, the secondary housing being adapted replaceably to receive a medicament cartridge

### FIELD OF INVENTION

The present invention relates to a medicament delivery device. Such medicament delivery devices include infusion devices and pen-type injectors. Such medicament delivery devices are typically used for self-administration of a medicament regimen by a patient, for example the self-administration of an insulin regimen by a person having diabetes.

### BACKGROUND TO THE INVENTION

In such medicament delivery devices the administration of the medicament is from a replaceable medicament cartridge located within a housing forming a part of the medicament delivery device. The medicament cartridge comprises a cylinder having medicament stored within a chamber defined by a moveable piston at a first end of the cartridge and a fluid impermeable membrane covering a second end of the cylinder.

One such known delivery device is the pen-type injector marketed under the name INNOVO by Novo Nordisk A/S. In this device the medicament cartridge is located within a locating breech, one end of which is located within a main housing of the injector. The medicament cartridge is loaded into the breech by inserting the first end of the cartridge into the main housing and subsequently lowering the second end into the breech. A secondary housing is then slid over the cartridge to hold the cartridge in position. Removal of the cartridge is effected by sliding the secondary housing away from the cartridge and then lifting the second end of the cartridge away from the breech. The cartridge may then be pulled away from the main housing.

This arrangement has as a disadvantage that removal of the cartridge is not easy for those with infirmities who are not able to grip the cartridge sufficiently firmly to place or remove the cartridge within the breech.

It is an advantage of the present invention that it provides an alternative means of loading and removing a medicament cartridge from a medicament delivery device. SUMMARY OF THE INVENTION

According to a first aspect of the present invention, a medicament delivery device comprises a main housing and a secondary housing, the secondary housing being pivotally connected at a first end to the main housing, the secondary housing being movable between an operative position in which the secondary housing is adjacent along its length to the main housing and a loading position in which the secondary housing is pivoted away from the main body, the secondary housing being adapted replaceably to receive a medicament cartridge.

Preferably, the main housing comprises drive means adapted to drive a piston within the medicament cartridge to expel medicament from within the cartridge in which locking means are provided to prevent movement of the secondary cartridge relative to the main housing while the drive means or a part thereof is located within the medicament cartridge to drive the piston.

### BRIEF DESCRIPTION OF THE DRAWINGS

The invention will now be described, by way of example only, with reference to the accompanying drawings, in which:

FIG. 1 shows a plan view of a first embodiment of a medicament delivery device in accordance with the present invention with the secondary housing in a first position;

FIG. 2 shows a view similar to FIG. 1 with the second housing in a second position; and

FIG. 3 shows a view similar to FIG. 2 showing a medicament cartridge to be inserted into the medicament delivery device of FIGS. 1 and 2.

### DETAILED DESCRIPTION OF THE INVENTION

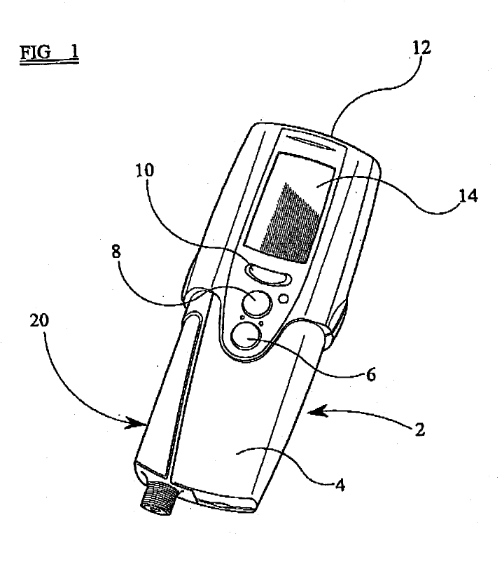

Referring first to FIGS. 1 to 3, there may be seen a first embodiment of a pen-type injector 2 in accordance with the present invention. The pen-type injector 2 comprises a main housing 4 about which are disposed a plurality of buttons 6, 8, 10, 12 by which a dose of medicament to be delivered may be selected, confirmed and subsequently be caused to be delivered, and a display 14 by which information, including information related to the operation of the injector, can be communicated to a user.

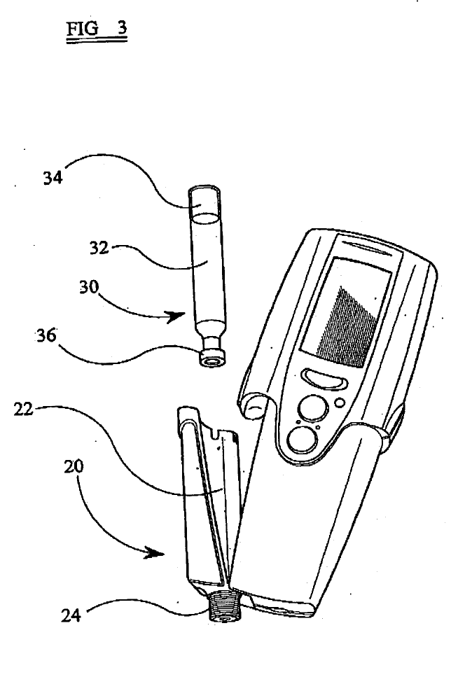

A medicament cartridge 30 for use with the medicament delivery device of the present invention is shown in FIG. 3. The medicament cartridge 30 comprises a cylinder 32 having medicament stored within a chamber defined by a moveable piston 32 at a first end of the cartridge and a fluid impermeable membrane covering a second end of the cylinder. The fluid impermeable membrane is conveniently secured in place by a mutual cap 36.

The injector 2 further comprises a secondary housing 20 connected for pivoting movement in relation to the main housing 4. The secondary housing 20 comprises a breech in the form of a sheath 22 into which the medicament cartridge 30 may be located and engagement means 24 to which a needle unit (not shown) may be secured.

Within the main housing 4 of the injector 2 there are conveniently located drive means (not shown) for moving a piston within a medicament cartridge, and a microprocessor (also not shown) for controlling operation of the drive means in response to the desired criteria selected by a user by way of the plurality of buttons 6, 8, 10, 12. It will be understood that as medicament is expelled from the medicament cartridge 30 the drive means, or at least a part thereof, will extend within the medicament cartridge 30 while the secondary housing 20 is in the first position shown in FIG. 1.

Conveniently, locking means or latching means may be provided releasably to secure the secondary housing in the first position.

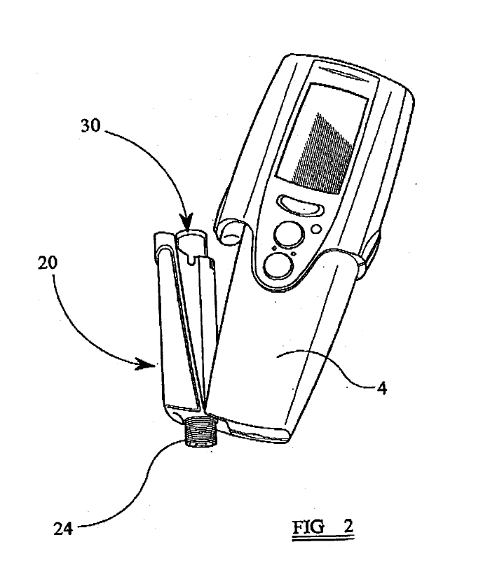

When it is desired to replace the medicament cartridge 30 within the injector 2, the drive means are caused to be withdrawn from within the medicament cartridge 30. The secondary housing 20 can then be rotated about the pivot by which the secondary housing 20 is secured to the main housing 4 until the secondary housing 20 is in the second position shown in FIGS. 2 and 3.

The medicament cartridge 30 may then be removed from the injector 2 either by grasping the second end of the medicament cartridge 30 between the fingers of a user and drawing the medicament cartridge 30 from the sheath 22. Alternatively, the user may simply invert the injector 2 and allow the medicament cartridge 30 to slide from within the sheath 22. A replacement medicament cartridge 30 may then be inserted or dropped into the sheath 22 before moving the secondary housing 20 back to the first position.

What is claims is:

1 A medicament delivery device comprising a main housing and a secondary housing, the secondary housing being pivotally connected at a first end to the main housing, the secondary housing being movable between an operative position in which the secondary housing is adjacent along its length to the main housing and a loading position in which the secondary housing is pivoted away from the main body, the secondary housing being adapted replaceably to receive a medicament cartridge.

2 A medicament delivery device according to claim 1, in which the main housing comprises drive means adapted to drive a piston within the medicament cartridge to expel medicament from within the cartridge in which locking means are provided to prevent movement of the secondary cartridge relative to the main housing while the drive means or a part thereof is located within the medicament cartridge to drive the piston.
